(19) United States
(12) Patent Application Publication
(10) Pub. No.: US 2011/0268798 A1
(43) Pub. Date: Nov. 3, 2011

(54) ORALLY DISNTEGRATING TABLETS FOR THE TREATMENT OF PAIN

(75) Inventors: Etienne Kraubsauer, Zimmersheim (FR); Frédéric Gerber, Blotzheim (FR); Burkhardt Sdchluetermann, Au (DE)

(73) Assignee: ADD ADVANCED DRUG DELIVERY TECHNOLOGIES LTD, REINACH (CH)

(21) Appl. No.: 13/138,131

(22) PCT Filed: Jan. 15, 2010
(86) PCT No.: PCT/EP2010/000213

§ 371 Date: Jul. 11, 2011

Related U.S. Application Data
(60) Provisional application No. 61/205,196, filed on Jan. 16, 2009.

(51) Int. Cl. A61K 31/616; A61P 25/00; A61K 9/20

(52) U.S. Cl. 424/465; 514/161; 424/464

(57) ABSTRACT

An orally disintegrating tablet comprising acetylsalicylic acid, acetaminophen and caffeine and one or more pharmaceutically acceptable excipients.

The present invention relates to orally disintegrating tablets, useful in particular for the treatment of pain, comprising a fixed dose combination of acetylsalicylic acid, acetaminophen, caffeine and corresponding manufacturing processes.

In an effort to develop more convenient dosage forms with an increased likelihood of improved compliance for certain product indications and patient populations, solid dosage forms are developed that can be ingested simply by placing them in the oral cavity, e.g. on the tongue. The products are designed to disintegrate rapidly on contact with saliva, thus eliminating the need to chew the tablet, swallow an intact tablet, or take the tablet with any liquids [7, 8, 9].

A fixed dose combination is a pharmaceutical preparation which contains one or more active pharmaceutical ingredients combined in a single dosage form presented in certain fixed doses. Typically, these fixed dose combination drug products offer benefits over the individually dosed single dose preparations, e.g. efficacy, dose reduction, ease of administration, safety, convenience, compliance.

A known fixed dose combination for the treatment of pain is the triple combination of acetylsalicylic acid, acetaminophen and caffeine. A triple combination of the above ingredients is also listed as a drug product along with specifications within USP 31; the monograph is entitled “Acetaminophen, Aspirin and Caffeine Tablets” [1].

Acetylsalicylic acid, also known as aspirin (USAN), is 2(acetyloxy)benzoic acid, C₉H₈O₄, with a molecular mass of 180.157 g/mol. Acetylsalicylic acid, CAS 50-78-2, appears as colourless or white crystals or white crystalline powder. Acetylsalicylic acid is slightly soluble in water, freely soluble in alcohol and soluble in chloroform and ether. Acetylsalicylic acid should be stored in airtight containers. The compound is stable in dry air but hydrolyses in contact with moisture to acetic and salicylic acids. Its pKₐ-value is 3.49. Acetylsalicylic acid exhibits a pH-dependent stability profile. The compound is sensitive to temperature as well. Acetylsalicylic acid has a slightly bitter and pronounced acidic taste. Acetylsalicylic acid is used as an analgesic to relieve minor aches and pains. Furthermore, the compound has an antipyretic effect, and is also used an anti-inflammatory medication. Due to its anti-clotting effect acetylsalicylic acid (aspirin) is also indicated in long-term treatment for prevention of heart attacks, strikes and blood clot formation [2].

Acetaminophen (USAN), also termed paracetamol, is N-(4-hydroxyphenyl)acetamide, C₈H₉NO₂, with a molecular mass of 151.169 g/mol. Acetaminophen, CAS 103-90-2, appears as white odourless crystalline powder, which is sparingly soluble in water, soluble 1 in 20 of boiling water, and in 1 in 10 of alcohol. The compound is very slightly soluble in ether and in methylene chloride. Its pKₐ-value is 9.38. The compound has a pronounced bitter taste. The drug substance is widely used as analgesic compound and antipyretic medication. In combination with non-steroidal anti-inflammatory drugs or opioid analgesics, acetaminophen is used also in the management of more severe pain [2].

Caffeine, which is 1,3,7-trimethyl-1H-purine-2,6(3H,7H)-dione, C₈H₁₀N₄O₂, with a molecular mass of 194.19 g/mol. Caffeine, CAS 58-08-2, appears as odourless, white needles or powder, which sublime readily. Caffeine is sparingly soluble in water and freely soluble in boiling water and in chloroform. Caffeine is slightly soluble in dehydrated alcohol and in ether. Its pKa-value is in the order of 0.6. The compound has a pronounced, long lasting, distinct bitter taste [2].

Drug products comprising these actives ingredients in a certain ratio are known for decades, e.g. in 1946 Germany's Dr. Karl Thomae GmbH developed Thomapyrin® and Bristol-Myers Squibb introduced its Excedrin® Extra Strength within the United States within the early 60ties. Both products are non-prescription, over-the-counter pain relievers [3, 4].

The current German Thomapyrin® drug product (Thomapyrin® classic) comprises 250 mg acetylsalicylic acid, 200 mg acetaminophen and 50 mg caffeine. The current marketed drug product is formulated as an immediate release tablet. Immediate release Excedrin® Extra Strength for the US market comprises 250 mg acetylsalicylic acid, 250 mg acetaminophen and 65 mg caffeine. In contrast to the European product, the US preparation contains slightly higher drug substance loads for acetaminophen and caffeine, i.e. 50 mg and 15 mg, respectively. In addition, the US product is formulated as film-coated tablet instead of a plain tablet.

Both formulations are fixed dose combinations where caffeine is used to increase the pain relieving effects of acetylsalicylic acid and acetaminophen. It was recently shown with Thomapyrin® that the fixed combination of acetylsalicylic acid, acetaminophen and caffeine is more effective than the individual substances and dual combinations for the treatment of headache [6]. The multi-centre, randomised, double-blind, single-dose, placebo-controlled parallel group study showed that for the primary endpoint “50% pain relief” in the intention-to-treat dataset (n=1743 patients), the fixed combination of acetylsalicylic acid, acetaminophen and caffeine was statistically significant superior to the combination without caffeine, the mono-substances acetylsalicylic acid and acetaminophen, caffeine and placebo. All active treatments except caffeine differed significantly from placebo. The authors also state that the triple combination showed superior efficacy for all secondary endpoints such as time until reduction of pain intensity, weighted sum of pain intensity difference, extent of impairment of daily activities, global assessment of efficacy.

In the course of time Thomapyrin® and Excedrin® brands have been extended. These new products are denominated line extensions and give customers a greater choice to identify the most suitable and convenient dosage form. For both products, Thomapyrin® and Excedrin®, the companies currently market several additional products under their already established brand name.

Classic Thomapyrin® pain relief tablets are currently accompanied by Thomapyrin® medium, which contains 400 mg acetaminophen and 50 mg of caffeine, Thomapyrin® intensiv, comprising 250 mg acetylsalicylic acid, 250 mg acetaminophen and 50 mg caffeine and Thomapyrin® effervescent tablets with 300 mg acetylsalicylic acid and 200 mg acetaminophen. Thomayprin® medium is also suited for customers with sensitivity towards acetylsalicylic acid. Thomapyrin® intensiv contains 50 mg more acetaminophen in comparison to Thomapyrin® classic and is therefore also suited to treat more severe aches and pain situations. Thomapyrin® effervescent tablets are an alternative pharmaceutical preparation lacking caffeine.

The current product palette of Excedrin drug products is even more diversified. Excedrin® extra strength is currently available in 4 different pharmaceutical presentations such as Excedrin® geltabs (gel-coated tablets), Excedrin® tablets, Excedrin® caplets (capsule shaped tablets) and fast-releasing Excedrin® express gels where headache relief starts 15 minutes after intake. All these products contain 250 mg acetylsalicylic acid, 250 mg acetaminophen and 65 mg caffeine.

Excedrin® PM (market introduction in 1969) combines in the forms of different pharmaceutical preparations 500 mg acetaminophen as pain reliever with 38 mg diphenhydramine citrate to promote steep. Excedrin® PM is now available as geltabs, tablets and caplets.

Excedrin® Migraine (1998) is a non-prescription pain reliever that contains 250 mg acetylsalicylic acid, 200 mg acetaminophen and 65 mg caffeine as well. The product is designed to treat migraine symptoms and is now marketed in three different dosage forms, i.e. geltabs, tablets and caplets.

Excedrin® Tension Headache (2003) provides fast relief for head, neck and shoulder pain associated with tension-type headaches and contains 500 mg acetaminophen along with 65 mg caffeine. The line extension is presented as geltabs, tablets, caplets and express gels.

Excedrin® Sinus Headache, introduced in 2005, is a non-prescriptive pain and nasal congestion reliever in the form of tablets or caplets which contain 325 mg acetaminophen as pain reliever and 5 mg phenylephrine hydrochloride as nasal decongestant.

Excedrin® Back and Body contains 250 mg acetaminophen and 250 mg acetylsalicylic acid buffered with calcium carbonate for fast and powerful relief of body aches. The product is formulated as a bilayered caplet and was introduced in 2007.

Over the past years there has been a growing demand for dosage forms which allow customers to administer their medication without the need of additional water/Liquid for flushing the drug product down.

Excedrin® Quick Tabs, an orally disintegrating tablet containing 500 mg acetaminophen and 65 mg, caffeine does currently not belong to the marketed Excedrin product palette.

However, even 60 years after the first market introduction there is no such dosage form, and in particular no oral disintegrating tablet, for the triple combination of acetylsalicylic acid, acetaminophen and caffeine, and in particular the triple combination of 250 mg acetylsalicylic acid, 200 mg acetaminophen and 50 mg caffeine or 250 mg acetylsalicylic acid, 250 mg acetaminophen and 65 mg caffeine on the market.

It is therefore an object of the present invention to provide such a solid dosage form with a combination of acetylsalicylic acid, acetaminophen and caffeine, and formulations and a corresponding manufacturing process for such a dosage form, in particular for those dosage forms combining 250 mg acetylsalicylic acid, 200 mg acetaminophen and 50 mg caffeine or 250 mg acetylsalicylic acid, 250 mg acetaminophen and 65 mg caffeine mainly for the treatment of pain.

The present invention provides an orally disintegrating tablet comprising acetylsalicylic acid, acetaminophen and caffeine and one or more pharmaceutically acceptable excipients.

According to the European Pharmacopoeia, these dosage forms, which are also referred to as orodispersible tablets, disperse readily before being swallowed. Orodispersible tablets are defined as disintegrating within 3 minutes in the European Pharmacopoeia [5]. This mode of administration is particularly beneficial for pediatric and geriatric patients, for people with conditions related to impaired swallowing, and for treatment of patients when compliance may be difficult (e.g., for psychiatric disorders). Orally disintegrating tablets are solid dosage forms which are especially advantageous for patients suffering from difficulties in swallowing medications. Others may prefer the discrete and easy way of administration of orally disintegrating dosage forms. Upon placement on the tongue these dosage forms disintegrate very rapidly, often within a matter of seconds.

Orally disintegrating tablets, as used herein, are solid oral preparations that disintegrate rapidly in the oral cavity (before being swallowed), with an in-vitro disintegration time of 3 minutes or less. Disintegration time is measured by the test method described below.

Orally disintegrating tablets according to this invention are tested for disintegration according to the European Pharmacopoeia, 2.9.1, with a disintegration tester, Type A, e.g. manufactured by Sotax, Basel, Switzerland. Apparatus A is designed for testing tablets up to a diameter of 18 mm. For larger tablets apparatus B can be used. [5].

Testing is performed according to the procedure outlined within the European Pharmacopoeia. One dosage form is placed in each tube of the disintegration basket. The apparatus is operated with demineralised water as disintegration medium of 37±2° C. as immersion fluid without discs. The tablets disintegrate rapidly. At the end of the test it is confirmed that all of the dosage units have disintegrated completely.

Orally disintegrating tablets according to this invention are designed to disintegrate within less than 3 minutes. Preferred embodiments disintegrate in less than 2 minutes, and even more preferably in less than 60 seconds.

Orally disintegrating tablets in the sense of the current invention are not limited with regard to the total amount of active ingredients contained therein. They may, in particular, contain more than 500 mg in total of active ingredients, a number mentioned in some references as an upper limit. For the combination of active ingredients of the ODTs of the present invention, the total amount of active ingredients may be in a range of from 150 mg to 1200 mg, for instance at least 200 mg, at least 300 or at least 400 mg, to name but a few examples. For instance, advantageous embodiments of the present invention comprise at least 500 mg in total of acetylsalicylic acid, acetaminophen and caffeine and thus provide a tablet that need not be swallowed whilst providing a suitably high dose of active ingredients, optionally with a pleasant taste.

A significant number of synonyms for orally disintegrating tablets is currently being used. Orally disintegrating tablets (ODTs) are also denominated orally dispersible tablets, orodispersible tablets, melt tablets, fast/rapid melt tablets, fast/rapid dissolving tablets, fast/rapid: dispersible tablets etc.

It is evident that the requirements that orally disintegrating tablets have to meet are significantly different from those of tablets configured to classical dosage form, e.g. immediate release tablets, which typically disintegrate in vivo after being swallowed (stomach) and tablets designed to be dissolved in water, e.g. effervescent tablets, to create a liquid dosage form.

Within the last years ODTs have become extremely popular as line extension products for prescription and non-prescription drug substances of different therapeutic categories, such as non-steroidal anti-inflammatory drugs, anti-ulcer drugs, antihistaminics, hypnotics, sedatives, antidepressants, antipsychotic drugs, antiparkinson drugs, antiemetics, analgesics including anti-migraine, antiemetics and drugs to treat erectile dysfunction. These drug products are especially useful for the treatment of acute and chronic symptoms and needs.

Nevertheless, formulation and process development work for ODTs is very challenging and extremely demanding since a multitude of cross functional aspects has to be considered and optimised. The tablet size should be minimized and the tablet matrix should disintegrate/dissolve practically immediately with minimal or no residue in the mouth. Insoluble particles should be easy to swallow and should have a small particle size. ODTs should be mechanically stable and should withstand packaging. The tablet should have a pleasant taste which may require taste-masking efforts if drug substances are bitter or have a poor taste. In addition, drug substance(s) and drug product(s) have to be stable. In most cases, the ODT line extension product should reveal a similar in vitro dissolution profile as the already marketed product. Furthermore, the ODT line-extension product may have to be bioequivalent to the brand product.

The development work for an OD tablet preparation is even more demanding, if high drug substance loads have to be incorporated into the tablet and/or different APIs (active pharmaceutical ingredients) have to be co-formulated as taste-masked preparation and is in particular challenging if the new line extension product must be bioequivalent to an existing marketed product.

The term “bioequivalence” and the attribute “bioequivalent” as used herein is related to bioavailability as follows. The term “bioavailability”, as used herein, is defined as a measure of the rate and amount of each active ingredient and/or corresponding key metabolite which reaches the systemic circulation following the administration of the dosage form.

The bioavailability of embodiments of the pharmaceutical oral fixed dose ODT of the present invention is compared herein with that of the corresponding standard reference product which is in the case of the triple combination cited above e.g. an immediate release (IR) solid dosage form, e.g. Thomapyrin® classic or Excedrin® Extra Strength. The test, the ODT fixed dose combination, and the reference, IR fixed dose reference product, formulations are administered orally to the subjects, and plasma samples are collected over time. The plasma samples are analyzed for concentration of APIs and/or key matabolites. The maximum plasma concentration (Cₘₐₓ) and the area under the plasma concentration vs. time curve (AUC) are calculated. Log-transformed AUC₀₋ₜₗₐₛₜ (AUC from time zero to the last measurable concentration sampling time), AUC₀₋₈ (AUC from time zero to infinity), Cₘₐₓ of each compound are analyzed separately using a suitable model, with fixed effects from sequence, treatment and period, and random effects from subject. A point estimate (ratio of geometric mean of Cₘₐₓ or AUC for test versus reference formulation) and the corresponding 90% confidence intervals are used to evaluate bioequivalence. For the test and reference products to be bioequivalent, the 90% confidence intervals for both AUC and Cₘₐₓ point estimates should fall within 0.8-1.25. Obtaining bioequivalence between test and reference products is challenging, particularly for fixed dose combinations and in particular for ODT versus IR products, and the results cannot be predicted a priori.

Whenever reference is made to an AUC being similar to the active ingredient in the reference product, it is meant that the AUC in the pharmaceutical oral fixed dose test combination of the present invention has preferably a 90% confidence interval which should fall within 0.8-1.25 for the active ingredients.

Whenever reference is made to a Cₘₐₓ being similar to the active ingredient in the reference product, it is meant that the Cₘₐₓ in the pharmaceutical oral fixed dose test combination of the present invention has preferably a 90% confidence interval which should fall within 0.8-1.25 for the active ingredients.

In a preferred embodiment, the pharmaceutical oral fixed dose ODT of the present invention has a release profile for one, both or all three active ingredients, such that the AUC and/or Cₘₐₓ point estimate(s) are in the range of from 0.8 to 1.3, more preferably of from 0.8 to 1.25, most preferably of from 0.85 to 1.2.

In another embodiment, the pharmaceutical oral fixed dose test combination of the present invention has a release profile for one, both or all three active ingredients, such that the 90% confidence interval for AUC(s) and/or Cₘₐₓ(s) are, of from 0.7 to 1.43, more preferably of from 0.7 to 1.30, still more preferably of from 0.75 to 1.25, most preferably of from 0.8 to 1.25.

It is preferred that at least the AUC(s), more preferably both the AUC(s) and the Cₘₐₓ(s) are within the above-mentioned ranges.

By virtue of this, the pharmaceutical fixed dose ODTs of the present invention can be configured so as to approach or preferably reach bioequivalence (if this is requested as target development profile for the product). Exemplary embodiments of the present invention are substantially bioequivalent to a corresponding immediate release product.

The orally disintegrating tablets according to the present invention can comprise 50-1000 mg acetylsalicylic acid, 50 to 1000 mg acetaminophen and 20-200 mg caffeine. Preferred are orally disintegrating tablets which comprise 250 mg acetylsalicylic acid, 200-250 mg acetaminophen, and 50-65 mg of caffeine.

In one embodiment, the orally disintegrating tablet according to the present invention comprises 250 mg acetylsalicylic acid, 200 mg acetaminophen and 50 mg caffeine. Such an ODT could serve as a line-extension product for the currently available European product.

In an alternative embodiment, the orally disintegrating tablet according to the present invention comprises 250 mg acetylsalicylic acid, 250 mg acetaminophen and 65 mg caffeine. Such an ODT could serve as a line-extension product for the currently available US product.

As described above, all three drug substances exhibit a bitter taste. Caffeine shows the most pronounced bitterness followed by acetaminophen and acetylsalicylic acid. The latter active ingredient exhibits a slightly bitter taste only, which is however accompanied by a distinct acidic sensation in the mouth.

Therefore, in a preferred embodiment, the ODT comprises a taste-masking agent. The taste-masking agent may comprise one or more components.

Taste-masking can be generally be achieved by addition of one or more sweeteners and flavours, for instance. Accordingly, in one embodiment, the taste-masking agent is a sweetener or flavour or a combination thereof.

Taste-masking can alternatively or additionally be achieved by encapsulating one or more of the active ingredients with a taste-masking agent. The active ingredient may be present in any suitable form, such as crystals, powders, micro-pellets prepared from those active ingredients, or any other suitable solid form.

Encapsulation can be achieved by film-coating, for instance. The active ingredient may be coated with a taste-masking agent that provides a taste-masking barrier. The resulting taste-mask coated active ingredient is also referred to herein as a micro-particle having or comprising a taste-masking coating in the following, or taste-masked microproduct, or taste-masked intermediate.

The taste-masking coating of the active ingredients should be able to withstand compression, since upon damage of the taste-masking coating, the preparation would immediately loose its taste-masking properties.

In a preferred embodiment of the orally disintegrating tablet according to the present invention, at least one of the active ingredients is provided with a taste-masking coating.

Preferably, the acetaminophen is contained in the form of micro-particles comprising a taste-masking coating, and/or the caffeine is contained in the form of micro-particles comprising a taste-masking coating.

In exemplary embodiments, the acetylsalicylic acid is contained in the form of micro-particles comprising a taste-masking coating.

Taste-masking agents useful in the present invention, and in particular suitable as taste-masking agents for the above active ingredients, comprise, by way of example, cellulose acetate (e.g CA 398-10), cellulose acetate butyrate (e.g. CAB-381-0.5), cellulose acetate phthalate (e.g. Aquacoat cPD), ethylcellulose (e.g. Ethocel 10), hypromellose phthalate (e.g. HPMCP), hypromellose acetate succinate (e.g. Aqoat), poly(butyl methacrylic, (2-dimethylaminoethyl)methacrylate, methyl methacrylate) 1:2:1 (e.g. Eudragit E 100, E 12.5, E PO), poly(ethyl acrylate, methyl methacrylate) 2:1 (e.g. Eudragit NE 30 D, NE 40 D), poly(methacrylic acid, methylmethacrylate) 1:1 (e.g. Eudragit L 100, L 12.5, L12.5 P), poly(methacrylic acid, ethyl acrylate) 1:1 (e.g. Acryl-EZE, Acryl-EZE MP, Eudragit L 30 D-55, L 100-55, Eastacryl 30D, Kollicoat MAE 30 D, MAE 30DP), poly(methacrylic acid, methyl methacrylate) 1:2 (Eudragit S 100, 12.5, 12.5 P), poly(methyl acrylate, methyl methacrylate, methacrylic acid) 7:3:1 (e.g. Eudragit FS 30D), poly(ethyl acrylate, methyl methacrylate, trimethylammonioethyl methacrylate chloride) 1:2:0.2 (e.g. Eudragit RL PO, RL 30 D, RL 12.5, RD 100), poly(ethyl acrylate, methyl methacrylate, trimethylammonioethyl methacrylate chloride) 1:2:0.1 (e.g. Eudragit RS 100, RS PO, RS 30 D, RS 12.5), polyvinyl acetate (Kollicoat SR 30 D), polyvinyl acetate phthalate (e.g. Sureteric) and any combination thereof. Preferably, ethylcellulose and cellulose acetate are used as a taste-masking agent.

With regard to the required amounts of polymers to be applied onto products to be taste-masked, the surface of the article to be coated has to be taken into account. Therefore, if APIs with an unpleasant taste have to be coated with a taste-masking barrier, the quantities are typically higher as if the taste-masking polymer has to be applied onto aggregated APIs and/or granules comprising these APIs. Furthermore, the coating quantities typically differ from API to API since bitterness and physico-chemical behaviour differ as well. Upon selection of the taste-masking polymer, the structure of the polymer and processing conditions have to be taken into account as well, e.g. molecular weight, degree of substitution, solvent used for processing, processing of melts etc. Furthermore, if too much taste masking compound is applied, drug release may be distinctly modified, i.e. drug release may be decreased.

In light of the present invention, the subsequent paragraphs comprise reasonable quantities which can be applied to mask the bitter taste and can subsequently be compressed into ODTs without changing the quality of the taste-masking properties.

Typical ranges of coating material applied for taste masking may be has high as 100% to be applied onto the active ingredients, preferrably only up to 60%, and even more preferrably a range of 5% up to 40%.

With regard to acetaminophen and acetylsalicylic acid 5-40% taste masking are preferred, more preferred is the application of 10-30%, and even more preferred is a taste masking with 20% of a coating.

With regard to caffeine 5-60% taste masking are preferred, more preferred is the application of 30-50%, and even more preferred is a taste masking with 40% of a coating.

The taste-masking materials are listed above. The preferred polymers according to this invention are, besides the above materials, ethylcelluose or cellulose acetate as taste masking film component.

These polymers may be combined with hydrophilic polymers and/or any other hydrophilic substance, such as e.g. hydroxypropyl cellulose, hydroxypropyl methylcellulose, hydroxyethyl cellulose, glucose, saccharose, povidone, copovidone, lactose, maize starch, citric acid to adjust drug release.

Accordingly, the taste-masking coating may comprise one or more taste-masking agents and optionally additional excipients, such as e.g. one or more release modifying excipients, as mentioned above.

Furthermore, the taste-masking agents may be combined with one or more suitable compounds which stabilize the active ingredients to be processed to a more suitable pH to guarantee long term chemical stability of the compound within an intermediate and/or final dosage form. In the case of acetyl salicylic acid, the taste-masking agent may be combined with a suitable amount of acid and/or basic compounds for pH-adjustment, e.g. but not limited to citric acid, tartaric acid, ascorbic acid and/or sodium carbonate, sodium bicarbonate and sodium phosphate. The pH-adjusting compounds for the orally disintegrating tablet according to this invention may as well be placed in the outer tablet phase as described below. In a preferred embodiment of this invention the orally disintegrating tablet contains 10-100 mg citric acid, in a more preferred embodiment the invention comprise 50 mg citric acid per tablet with a tablet mass of 1200 mg. Expressed differently, in one embodiment of this invention the orally disintegrating tablet contains 0.8 to 8 wt % citric acid, based on the total weight of the tablet.

Since the final particle size of the active ingredient, and in case of a taste-masked active ingredient the final size of the sufficiently taste-masked micro-particles within an ODT formulation is crucial e.g. with regard to acceptable mouth feel and dissolution performance, it is desirable that the particle size of the API raw material be minimised.

Therefore, the following qualities of raw materials of the active ingredients are preferably employed in accordance with the present invention; for characterisation purposes standard hand sieving equipment (e.g. Retsch) or air jet sieving equipment (e.g. Alpine) can be used.

For acetylsalicylic acid, a quality wherein =1 wt. % of the raw material are retained on a 250 μm sieve, and =40 wt. % on a 63 μm sieve has been found to be advantageous. For acetaminophen, a quality wherein =5 wt. % of the raw material are retained on a 250 μm sieve and =30 wt. % of the raw material on a 63 μm sieve has been found to be advantageous. For caffeine, a quality wherein 97 wt. % of the raw material pass a 150 μm sieve has been found to be advantageous.

In exemplary embodiments of the present invention, encapsulation of active pharmaceutical ingredients with a taste-masking barrier is carried out with fluid-bed equipment and is performed as top spray coating process, or alternatively as Wurster coating procedure.

The resulting taste-masked micro-particles can have a particle size less than 500 μm and are in the range of approximately 50-400 μm with a mean particle size in the range of 100-250 μm. More preferably the taste-masked micro-particles have a particle size in the range of 50-315 μm with a mean particle size in the range of range of 100-150 μm.

For the ease and convenience of administration of an orally disintegrating tablet, these dosage forms should in principal be minimized in size and mass. This is in particular challenging if fixed combinations have to be realised, if the drug substance loads are high, if taste-masking has to be achieved and if means to stabilise drug substances have to be considered in parallel.

Therefore, orally disintegrating tablets according to the present invention exhibit a tablet mass of maximally 2000 mg. Preferred are orally disintegrating tablets with a tablet mass of <1500 mg, and even more preferred are dosage forms with a tablet mass of =1300 mg.

The one or more excipients to be used in the ODT according to the present invention may include the following:

a) one or more fillers, e.g. and without limitations, micro-crystalline cellulose, mannitol, sorbitol, xylitol, glucose, sugar and sugar derivates, calcium hydrogen phosphates, and mixtures thereof,

b) one or more binders, e.g. and without limitations, hyprodroxypropyl methylcellulose, hydroxypropyl cellulose, methylcellulose, hydroxymethyl cellulose, hydroxyethyl cellulose, alginates, pektines, polyvinylacetate, povidone and copovidone, polyvinypyrrolidone, xanthan gum, polyvinyl alcohol, starch and pregelatinized starch and mixtures thereof,

c) one or more glidants, e.g. and without limitations, talc, silicon dioxide, colloidal silicon dioxide, and mixtures thereof,

d) one or more disintegrants, e.g. and without limitations, crospovidone, cross-linked sodium carboxymethylcellulose, sodium starch glycolate, starch, pregelatinised starch, and mixtures thereof,

e) one or more sweeteners, e.g. and without limitations, acesulfame potassium, alitame, aspartame, cyclamate, glycyrrhizin, saccharin, sucralose, sugar, inulin and mixtures thereof,

f) one of more flavours, e.g. and without limitations, lemon, orange, cola, mandarine, tangerine, grapefruit, cherry, vanilla, strawberry, raspberry, peppermint flavours and mixtures thereof

g) one or more colourants, e.g. and without limitations yellow, orange, red, green, and blue, colourants and mixtures thereof,

h) one ore more lubricants, e.g. and without limitations, magnesium stearate, hydrogenated castor oil, hydrogenated vegetable oil, stearic acid, sodium stearyl fumarate, macrogol, talc, sugar alcohols and mixtures thereof to form orally disintegrating tablets upon compression.

a), b), d) can be added in the formulation also in a pre-dried form to reduce the internal moisture in the final tablet. Pre-drying of the excipients can be performed prior to processing as a separate manufacturing step, but it is also possible to use pre-dried excipients sourced directly from the vendor.

c), h) excipients do not have necessarily to be applied within the blend for tabletting. These components can also be applied externally during the compression step directly to punch heads and dies. This application technology is in the case of the use of a lubricant known as external lubrication.

In a preferred embodiment, the orally disintegrating tablet comprises micro-particles comprising mannitol and a disintegrant as an excipient. The micro-particles may consist of mannitol and a disintegrant, i.e. contain no further components. In another embodiment, the micro-particles may contain mannitol and two or more disintegrants, optionally with additional components. Spherical particles have been found to be particularly advantageous. A preferred embodiment therefore comprises spherical particles of mannitol and a disintegrant.

The disintegrant may be selected from crospovidone, sodium starch glycolate, cross-linked sodium carboxymethyl cellulose and any mixture thereof, for instance.

In an exemplary embodiment, the ODT comprises spherical particles comprising, preferably consisting of, mannitol and a disintegrant selected from crospovidone, sodium starch glycolate, cross-linked sodium carboxymethyl cellulose and any mixture thereof. In one embodiment, a combination of mannitol and crospovidone is used. In another embodiment, a combination of mannitol and sodium starch glycolate is used. A further embodiment involves use of a combination of cross-linked sodium carboxymethyl cellulose and mannitol.

Additional excipients, e.g. stabilizers to adjust for a certain pH or low amounts of viscosity increasing agents (see binders) to adjust the consistency of the disintegrating tablet within the mouth, may be incorporated into the tablet formulation, as well. Stabilizers include, without limitations, citric acid, tartaric, ascorbic acid, sodium carbonate, sodium bicarbonate, sodium phosphates and mixtures thereof.

For compression, standard compression machines, e.g. single station presses or rotary tablet machines, can be used (e.g. Korsch, Fette, Kikusui). The compression is, for instance, carried out such that a crushing strength in the range of 10 to 200 N is achieved. The crushing strength is determined in accordance with the European Pharmacopeia, Volume 6.0, 2.9.8, with a tablet hardness tester manufactured by Schleuninger, Switzerland: [5]. Since the crushing strength exerts an influence on disintegration, exemplary embodiments have a crushing strength of at least 10 N, for instance in a range of 10 to 200 N, for instance between 10 and 100 N.

The friability is determined in accordance with the European Pharmacopeia, Volume 6, 2.9.7., with a friability tester manufactured by Sotax, Switzerland [5]. Since the friability is an indicator on mechanical stability of the orally disintegrating tablet, exemplary embodiments have a friability of at least 4.5%, for instance in the range of <1 to 5%, for instance between <1 and 4%, for instance 3.5% and less. No cracked, cleaved or broken tablets are present in the tablet sample after tumbling.

As a filler or filler combination for the orally disintegrating tablets according to the present invention, water soluble carbohydrates may advantageously be used. A suitable filler or filler combination may, for instance, be selected from mannitol (e.g. Pearlitol 160 C from Roquette), sorbitol, glucose, and any combination thereof, as mentioned before.

In a preferred embodiment of the invention the tablets contain glucose in a range of 25-250 mg. More preferred is an orally disintegrating tablet with an amount of glucose of 25-100 mg. Even more preferred is a formulation for an orally disintegrating tablet concerning this invention with an amount of 60 mg glucose.

As mentioned before, in a preferred embodiment of this invention, spherical particles consisting of a disintegrant, e.g. crospovidone and/or sodium starch glycolate and/or cross-linked sodium carboxymethyl cellulose, and mannitol are employed as fillers for ODT formulations. Such formulations have the advantage that the disintegrant does not just serve as disintegrant, but also provides a water reservoir during processing (see below) which facilitates the spheronisation of the carbohydrate and thus leads to end product having advantageous properties.

These spherical particles may be prepared by blending crospovidone (such as Kollidon CL-M of BASF) and/or sodium starch glycolate (such as Explotab of JRS or Edward Mendell of DMV) and/or cross-linked sodium carboxymethyl cellulose (Ac-Di-Sol from by FMC) together with mannitol in a vertical granulator, homogeneously moistening the blend with an aqueous solution of mannitol in water, and spheronising the prewetted material in suitable fluidised bed direct pelletisation equipment. Suitable processing conditions are provided in one of the exemplary embodiments.

The quality of orally disintegrating tablets is assessed according to the pharmacopoeial tests/methods with conventional testing equipment including e.g. assay, content uniformity, purity, disintegration time, dissolution behaviour, loss on drying, crushing strength and friability [1,5].

Dissolution testing of the ODTs is performed using an USP Type II dissolution apparatus (paddle) operating at 100 rpm in 900 ml of water at 37° C. The cumulative drug releases are determined offline by HPLC-quantification. Determination of identity, assay and related substances is performed on 10 tablets by HPLC with a Nucleosil column (C18 pack, pore size 5 μm, length 125 mm, internal diameter 4.0 mm) and a pre-column (C18 pack, pore size 5 μm, length 4 mm, internal diameter 3.0 mm), using an isocratic method (630 ml Phosphate buffer pH 2.5 mixed with 370 ml methanol) with a flow rate of 1.0 ml/min at a column temperature of 40° C.

The total run time is 6 min. For assay and identity the injection volumes are 2 μl of test and reference solutions, equivalent to about 1.0 μg of caffeine, 4 μg of acetaminophen and 5 μg of acetylsalicylic acid. Quantification is performed at a wavelength of 275 nm. For evaluation of the active ingredient degradation the injection volumes are increased to 5 μl of test and reference solutions, equivalent to about 0.037 μg of salicylic acid. The samples are evaluated at a wavelength of 230 nm.

With regard to taste the orally disintegrating tablets can be assessed by a taste panel according to the procedure outlined in the European Pharmacopoeia 6.0, 2.8.15., “Bitterness Value”, against solutions of different quinine hydrochloride concentrations in water as reference [5]. Sample (test) preparation can be performed by dispersing an orally disintegrating tablet in 100 ml of water of 37° C. After 1 minute with gentle stirring any remaining particles are filtered off. The remaining filtered solution has a dilution factor (df) of 100. The examination can be performed as outlined within the monography.

In addition, to the test to evaluate the bitterness value, the remaining solution described above can be analysed quantitatively by HPLC with regard to its major bitter compounds, i.e. acetaminophen and caffeine, and can be compared to an orally disintegrating tablet formulation prepared with non taste-masked active ingredients.

Accelerated and long term stability studies are performed in white duma twist-off plastic container (30 mL) made out of high-density polyethylene (HDPE), with a threaded neck with duma twist-off cap (not sealed) including a desiccant (1.25 g). The bottles containing stability samples are placed in different climatic chamber at the following storage conditions:
25° C./60% relative humidity (rh)
30° C./65% rh

The invention further provides a method for preparing an orally disintegrating tablet according to the present invention, comprising

a) blending the acetylsalicylic acid, acetaminophen and caffeine with one or more pharmaceutically acceptable ingredients, and

b) compressing the resulting blend into tablets.

In a further embodiment, the method further comprises applying a taste-masking coating to the acetylsalicylic acid and/or acetaminophen and/or caffeine before step a). After application of the taste-masking coating, the intermediates (micro-particles) are combined with the one or more suitable pharmaceutically acceptable excipients to form orally disintegrating tablets upon compression.

The present invention further concerns the use of acetylsalicylic acid, acetaminophen and caffeine for preparing a medical composition in the form of an orally disintegrating tablet in particular for treating pain. The invention further relates to preparations and corresponding manufacturing processes of the said compounds, which are suitable to be used as a treatment for pain.

In the following, the invention will be described with reference to exemplary embodiments. The abbreviation TM or tm, as used in the following, stands for “taste-masked”.

### EXAMPLE 1

### tm Caffeine, tm Acetaminophen, Acetylsalicylic Acid, Mannitol

Acetaminophen is taste-masked with Ethocel 10 (pure ethylcellulose from Dow/Colorcon) by top-spray processing or Wurster coating (Glatt GPCG 1). Application of the taste-masking agent Ethocel 10 is performed by spraying 1.5 kg ethanol solution containing 10% w/w Ethocel 10 onto 750.0 g acetaminophen using a spray rate of 25 g/min and 30° C. product temperature. After drying (10 minutes at 60° C. inlet air temperature), the taste-masked acetaminophen exhibits a narrow particle size distribution ranging from 50 to 315 μm, with a mean particle size of approximately 125 μm.

The taste-masking of caffeine is performed following the same processing conditions as described above for acetaminophen using a solution of ethanol/water 95:5 containing 10% w/w Ethocel 10. In this case, the application is performed by spraying 3.0 kg ethanol/water 95:5 solution containing 10% w/w Ethocel 10 onto 750.0 g caffeine with a spray rate of 22-25 g/min and 30° C. product temperature. After drying (10 minutes at 60° C. inlet air temperature), the taste-masked caffeine exhibits a narrow particle size distribution ranging from 50 to 315 μm, with a mean particle size of approximately 175 μm and more preferably 125 μm.

The obtained taste-masked micro-products (acetaminophen and caffeine) are combined with acetylsalicylic acid, mannitol, maize starch, sodium starch glycolate, cross-linked carboxymethlycellulose sodium or crospovidone, silicon dioxide, comminuted sucralose, a flavor and eventually pigments, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK0 instrumented single station press equipped with 18 mm round, biplane punches, with bevelled edges. The obtained ODTs exhibit an average mass of ˜1200 mg, a crushing strength of ˜40 N and an average height of ˜5 mm.

The disintegration time of the orally disintegrating tablets as described above determined in water according to the method outlined in European Pharmacopoeia, Vol. 6, 2.9.1., Apparatus A, is below 60 seconds.

a) Formulation A

| | Amount | |
|---|---|---|
| Component | (mg/tablet) | (%) |
| TM Acetaminophen | 240.00 | 20.00 |
| (200 mg acetaminophen with | | |
| 40 mg ethylcelulose) | | |
| Acetylsalicylic acid | 250.00 | 20.83 |
| TM Caffeine | 70.00 | 5.83 |
| (50 mg caffeine with | | |
| 20 mg ethylcellulose) | | |
| Mannitol | 496.00 | 41.33 |
| Maize starch | 60.00 | 5.00 |
| Crospovidone (other alternative | 36.00 | 3.00 |
| is sodium starch glycolate or | | |
| cross-linked carboxymethlycellulose sodium) | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

b) Formulation B

| | Amount | |
|---|---|---|
| Component | (mg/tablet) | (%) |
| TM Acetaminophen (250 mg acetaminophen with | 300.00 | 25.00 |
| 50 mg ethylcellulose) | | |
| Acetylsalicylic acid | 250.00 | 20.83 |
| TM Caffeine (65 mg caffeine with 26 mg | 91.00 | 7.58 |
| ethylcellulose) | | |
| Mannitol | 415.00 | 34.58 |
| Maize starch | 60.00 | 5.00 |
| Crospovidone (other alternative is sodium starch | 36.00 | 3.00 |
| glycolate or cross-linked carboxymethlycellulose | | |
| sodium) | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

For both formulations A and B, the ODTs exhibit complete acetylsalicylic acid drug release after 5 minutes when tested using an USP Type II dissolution apparatus (paddle) operating at 100 rpm in 900 ml of water at 37° C. Acetaminophen and caffeine releases from the above ODTs are above 80% after 15 minutes when tested following the same method as described for acetylsalicylic acid.

### EXAMPLE 2

### tm Caffeine, tm Acetaminophen, tm Acetylsalicylic Acid, Mannitol

Taste-masking of acetaminophen and caffeine is performed separately following the same processing conditions as described above in example 1.

Acetylsalicylic acid is taste-masked with Ethocel 10/HPC-L 80:20 by top-spray processing or Wurster-Coating (Glatt GPCG 1). HPC-L is hydroypropyl cellulose from Nisso. The application of the taste-masking barrier is performed by spraying 1.5 kg of ethanol solution containing 8% w/w Ethocel 10 and 2% HPC-L onto 750.0 g acetylsalicylic acid with a spray rate of 25 g/min and 30° C. product temperature. After drying (15 minutes at 60° C. inlet air temperature), the taste-masked acetylsalicylic acid exhibits a narrow particle size distribution ranging from 50 to 315 μm, with a mean particle size of approximately 1.50 μm and more preferably 125 μm.

The obtained taste-masked micro-products (acetaminophen, caffeine and acetylsalicylic acid) are combined with mannitol, maize starch, sodium starch glycolate, cross-linked carboxymethylcellulose sodium or crospovidone, silicon dioxide, comminuted sucralose, a flavor and eventually colorants, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK0 instrumented single station press equipped with 18 mm round, biplane punches, with bevelled edges. For Formulation C, the obtained ODTs exhibit an average mass of ˜1200 mg, a crushing strength of ˜40 N and an average height of ˜5 mm. For Formulation D, the obtained ODTs exhibit an average mass of ˜1300 mg, a crushing strength of ˜40 N and an average height of ˜5.5 mm.

The disintegration time of the orally disintegrating tablets as described above determined in water according to the method outlined in a) European Pharmacopoeia, Vol. 6, 2.9.1., Apparatus A, is below 60 seconds.

a) Formulation C

| | Amount | |
|---|---|---|
| Component | (mg/tablet) | (%) |
| TM Acetaminophen (200 mg acetaminophen with | 240.00 | 20.00 |
| 40 mg ethylcellulose) | | |
| TM Acetylsalicylic acid (250 mg acetylsalicylic | 300.00 | 25.00 |
| acid with 40 mg ethylcellulose and 10 mg | | |
| hydroxypropycellulose) | | |
| TM Caffeine (50 mg caffeine with 20 mg | 70.00 | 5.83 |
| ethylcellulose) | | |
| Mannitol | 446.00 | 37.17 |
| Maize starch | 60.00 | 5.00 |
| Sodium starch glycolate (other alternative is | 36.00 | 3.00 |
| cross-linked carboxymethylcellulose sodium | | |
| or crospovidone | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

b) Formulation D

| | Amount | |
|---|---|---|
| | (mg/ | |
| Component | tablet) | (%) |
| TM Acetaminophen (250 mg acetaminophen with | 300.00 | 23.08 |
| 50 mg ethylcellulose) | | |
| TM Acetylsalicylic acid (250 my acetylsalicylic acid | 300.00 | 23.08 |
| with 40 mg ethylcellulose and 10 mg | | |
| hydroxypropyl cellulose) | | |
| TM Caffeine (65 mg caffeine with 26 mg | 91.00 | 7.00 |
| ethylcellulose) | | |
| Mannitol | 453.00 | 34.85 |
| Maize starch | 65.00 | 5.00 |
| Sodium starch glycolate (other alternative is | 39.00 | 3.00 |
| cross-linked carboxymethylcellulose sodium or | | |
| crospovidone | | |
| Silicon dioxide | 26.00 | 2.00 |
| Stearic acid | 6.50 | 0.50 |
| Sucralose | 6.50 | 0.50 |
| Flavor | 13.00 | 1.00 |
| Total | 1300.00 | 100.00 |

For both formulations C and D, acetylsalicylic acid drug release from the ODTs is above 70% after 15 minutes when tested using an USP Type II dissolution apparatus (paddle) operating at 100 rpm in 900 ml of water at 37° C. Acetaminophen and caffeine releases from the ODTs are above 80% after 15 minutes when tested following the same method as described for acetylsalicylic acid.

### EXAMPLE 3

### tm Caffeine, tm Acetaminophen, Acetylsalicylic Acid, Spherical Mannitol Particles

Taste-masking of acetaminophen and caffeine is performed separately following the same processing conditions as described above in example 1.

The manufacture of spheronised mannitol products is performed by direct pelletisation. After pre-wetting of 1.25 kg from a mixture of 90% w/w Mannitol and 10% w/w crospovidone or sodium starch glycolate in a high shear granulator (Glatt VG 10) by application of 200.0 g saturated Mannitol solution (15% w/w solids), the pre-wetted material is then equilibrated by a subsequent sieving step. Spheronisation of the pre-wetted blend is performed using a Glatt CPS 3 instrument by spraying 500.0 g purified water (spray rate 20 g/min) directly onto the product using a binary nozzle. After drying within a Glatt GPCG 1 (20 minutes at 60° C. inlet air temperature), the product exhibits a narrow particle size distribution, ranging from 200 to 500 μm, as well as a smooth and uniform surface.

The obtained taste-masked micro-products (acetaminophen and caffeine) are combined with acetylsalicylic acid, the spheronised mannitol product, sodium starch glycolate, cross-linked carboxymethlycellulose sodium or crospovidone, silicon dioxide, comminuted sucralose, a flavor and eventually pigments, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK0 instrumented single station press equipped with 18 mm round, biplane punches, with bevelled edges. The obtained ODTs exhibit an average mass of ˜1200 mg, a crushing strength of ˜40 N and an average height of ˜5 mm.

The disintegration time of the orally disintegrating tablets as described above determined in water according to the method outlined in a) European Pharmacopoeia, Vol. 6, 2.9.1., Apparatus A, is below 60 seconds.

a) Formulation E

| | Amount | |
|---|---|---|
| Component | (mg/tablet) | (%) |
| TM Acetaminophen (200 mg acetaminophen | 240.00 | 20.00 |
| with 40 mg ethylcellulose) | | |
| Acetylsalicylic acid | 250.00 | 20.83 |
| TM Caffeine (50 mg caffeine with 10 mg | 70.00 | 5.83 |
| ethylcellulose) | | |
| Mannitol spheres with crospovidone or with | 556.00 | 46.33 |
| sodium starch glycolate | | |
| Crospovidone (other alternative is sodium starch | 36.00 | 3.00 |
| glycolate or cross-linked carboxymethlycellulose | | |
| sodium) | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

b) Formulation F

| | Amount | |
|---|---|---|
| Component | (mg/tablet) | (%) |
| TM Acetaminophen (250 mg acetaminophen | 300.00 | 25.00 |
| with 50 mg ethylcellulose) | | |
| Acetylsalicylic acid | 250.00 | 20.83 |
| TM Caffeine (65 mg caffeine with 26 mg | 91.00 | 7.58 |
| ethylcellulose) | | |
| Mannitol spheres with crospovidone or with | 475.00 | 39.58 |
| sodium starch glycolate | | |
| Crospovidone (other alternative is sodium starch | 36.00 | 3.00 |
| glycolate or cross-linked carboxymethlycellulose | | |
| sodium) | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

For both formulations E and F, the ODTs exhibit complete acetylsalicylic acid drug release after 5 minutes when tested using an USP Type II dissolution apparatus (paddle) operating at 100 rpm in 900 ml of water at 37° C. Acetaminophen and caffeine releases from the ODTs are above 80% after 15 minutes when tested following the same method as described for acetylsalicylic acid.

### EXAMPLE 4

### tm Caffeine, tm Acetaminophen, tm Acetylsalicylic Acid, Spherical Mannitol Particles

Taste-masking of acetaminophen, caffeine and acetylsalicylic acid are performed separately following the same processing conditions as described above in examples 1 and 2. The manufacture of spheronised mannitol is performed following the same processing conditions as described in example 3.

The obtained taste-masked micro-products (acetaminophen, caffeine and acetylsalicylic acid) are combined with mannitol spheres with sodium starch glycolate or crospovidone, sodium starch glycolate, cross-linked carboxymethylcellulose sodium or crospovidone, silicon dioxide, comminuted sucralose, a flavor and eventually pigments, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK 0 instrumented single station press equipped with 18 mm round, biplane punches, with bevelled edges.

The disintegration time of the orally disintegrating tablets as described above determined in water according to the method outlined in European Pharmacopoeia, Vol. 6, 2.9.1., Apparatus A, is below 60 seconds,

a) Formulation G

| | Amount | |
|---|---|---|
| Component | (mg/tablet) | (%) |
| TM Acetaminophen (200 mg acetaminophen | 240.00 | 20.00 |
| with 40 mg ethylcellulose) | | |
| TM Acetylsalicylic acid (250 g acetylsalicylic | 300.00 | 25.00 |
| acid with 40 mg ethylcellulose and 10 mg | | |
| hydroxypropyl cellulose) | | |
| TM Caffeine (50 mg caffeine with 20 mg | 70.00 | 5.83 |
| ethylcellulose) | | |
| Mannitol spheres with sodium starch glycolate or | 506.00 | 42.17 |
| with crospovidone | | |
| Sodium starch glycolate (other alternative is | 36.00 | 3.00 |
| cross-linked carboxymethylcellulose sodium | | |
| or crospovidone) | | |
| Silicon dioxide | 24.00 | 2.00 |
| Stearic acid | 6.00 | 0.50 |
| Sucralose | 6.00 | 0.50 |
| Flavor | 12.00 | 1.00 |
| Total | 1200.00 | 100.00 |

For Formulation G, the obtained ODTs exhibit an average mass of ˜1200 mg, a crushing strength of ˜40 N and an average height of ˜5 mm.

b) Formulation H

| | Amount | |
|---|---|---|
| Component | (mg/tablet) | (%) |
| TM Acetaminophen (250 mg acetaminophen | 300.00 | 23.08 |
| with 50 mg ethylcellulose) | | |
| TM Acetylsalicylic acid (250 mg acetylsalicylic | 300.00 | 23.08 |
| acid, 40 mg ethylcellulose, 10 mg | | |
| hydroxypropyl cellulose) | | |
| TM Caffeine (65 mg caffeine and 26 mg | 91.00 | 7.00 |
| ethylcellulose) | | |
| Mannitol spheres with sodium starch glycolate or | 518.00 | 39.85 |
| with crospovidone | | |
| Sodium starch glycolate (other alternative is | 39.00 | 3.00 |
| cross-linked carboxymethylcellulose sodium | | |
| or crospovidone) | | |
| Silicon dioxide | 26.00 | 2.00 |
| Stearic acid | 6.50 | 0.50 |
| Sucralose | 6.50 | 0.50 |
| Flavor | 13.00 | 1.00 |
| Total | 1300.00 | 100.00 |

For Formulation H, the obtained ODTs exhibit an average mass of ˜1300 mg, a crushing strength of ˜40 N and an average height of ˜5.5 mm.

For both formulations G and H, acetylsalicylic acid drug release from the ODTs is above 70% after 15 minutes when tested using an USP Type II dissolution. apparatus (paddle) operating at 100 rpm in 900 ml of water at 37° C. Acetaminophen and caffeine releases from the ODTs are above 80% after 15 minutes when tested following the same method as described for acetylsalicylic acid.

### EXAMPLE 5

For preparation of these spherical particles, crospovidone (e.g. Kollidon CL-M of BASF) and/or sodium starch glycolate (e.g. Explotab of JRS or Edward Mendell, Primojel of DMV) and/or cross-linked sodium carboxymethyl cellulose (Ac-Di-Sol from by FMC) are blended together with mannitol (e.g. Pearlitol 25 C from Roquette) within a suitable vertical granulator, e.g. VG 10, Fa. Glatt, in a ratio of 10:90 (m:m). Subsequently the blend may be homogenously moistened with an aqueous solution of mannitol in water, the amount of solids in the solution is 15%. The residual moisture of the prewetted product is approximately 10% (Mettler Halogen Moisture Balance, mass approximately 5 g, 105° C., 1 mg/30 sec). After optionally passing the material through a sieve with an aperture width of approximately 3.15 mm, the prewetted material is being spheronised upon spraying of demineralised water within the fluidised bed direct pelletisation equipment CPS of Glatt until the mean particle size of the spheres is around 200 μm. Therefore, the CPS is equipped with a 30° rotor plate and four 4 flat baffles for direction of the pellet bed. The inlet air temperature during spheronisation is set to 35° C. The inlet air volume amounts to 75 m³/h. The rotor speed is set to 350 rpm. The residual moisture determined as loss on drying of the spheronised product is approximately 20% and is determined using the method described above. The spheres are subsequently dried in a standard fluid bed coater, e.g. GPCG 1, GPCG 3, Fa. Glatt, operating at an inlet air temperature of 60° C. The product is dried until the residual moisture of the dried material reaches values <0.2%. The bulk density of the spherical particles is approximately 0.6 g/ml.

### EXAMPLE 6

### tm Caffeine, tm Acetaminophen, tm Acetylsalicylic Acid, Mannitol

Acetaminophen is taste-masked with CA 398-10 (pure cellulose acetate from Eastman) by top-spray processing or Wurster coating (Glatt GPCG 1). Application of the taste-masking agent CA 398-10 is performed by spraying 1.5 kg acetone/water 95:5 solution containing 10% w/w CA 398-10 onto 750.0 g acetaminophen using a spray rate of 22-24 g/min and 25° C. product temperature. After drying (30 minutes at 60° C. inlet air temperature), the taste-masked acetaminophen exhibits a narrow particle size distribution ranging from 50 to 315 μm, with a mean particle size of approximately 125 μm.

The taste-masking of caffeine is performed following the same processing conditions as described above for acetaminophen using a solution of acetone/water 95:5 containing 10% w/w CA 398-10. In this case, the application is performed by spraying 3.0 kg acetone/water 95:5 solution containing 10% w/w CA 398-10 onto 750.0 g caffeine with a spray rate of 30 g/min and 25° C. product temperature. After drying (30 minutes at 60° C. inlet air temperature), the taste-masked caffeine exhibits a narrow particle size distribution ranging from 50 to 315 μm, with a mean particle size of approximately 125 μm.

Acetylsalicylic acid is taste-masked using a solution of acetone/water 95:5 containing 1.0% w/w CA 398-10 by top-spray processing or Wurster-Coating (Glatt GPCG 1). The application of the taste-masking barrier is performed by spraying 1.125 kg of acetone/water 95:5 solution containing 10% w/w CA 398-10 onto 750.0 g acetylsalicylic acid with a spray rate of 20 g/min and 25° C. product temperature. After drying (30 minutes at 60° C. inlet air temperature), the taste-masked acetylsalicylic acid exhibits a narrow particle size distribution ranging from 50 to 250 μm, with a mean particle size of approximately 125 μm.

The obtained taste-masked micro-products (acetaminophen, caffeine and acetylsalicylic acid) are combined with mannitol, maize starch, crospovidone, sodium starch glycolate or cross-linked carboxymethylcellulose sodium, talc, silicon dioxide, comminuted sucralose, eventually flavors and pigments, and pre-blended during 15 minutes in a Turbula T2C diffusion blender. After pre-blending, stearic acid is added to the pre-blend and blending is continued: with the same equipment for additional 5 minutes.

Compression of laboratory scale batches is performed with a Korsch EK0 instrumented single station press equipped with 18 mm round, biplane punches, with bevelled edges. For Formulation I, the obtained ODTs exhibit an average mass of ˜1200 mg, a crushing strength of ˜40 N and an average height of ˜5 mm. For Formulation J, the obtained ODTs exhibit an average mass of ˜1300 mg, a crushing strength of ˜40 N and an average height of ˜5.5 mm.

The disintegration time of the orally disintegrating tablets as described above determined in water according to the method outlined in a) European Pharmacopoeia, Vol. 6, 2.9.1., Apparatus A, is below 60 seconds.

a) Formulation I

| | Amount | |
|---|---|---|
| Component | (mg/tablet) | (%) |
| TM Acetaminophen (200 mg acetaminophen | 240.00 | 20.00 |
| with 40 mg cellulose acetate) | | |
| TM Acetylsalicylic acid (250 g acetylsalicylic | 287.50 | 23.96 |
| acid with 37.5 mg cellulose acetate) | | |
| TM Caffeine (50 mg caffeine with 20 mg | 70.00 | 5.83 |
| cellulose acetate) | | |
| Mannitol | 416.50 | 34.71 |
| Maize starch | 120.00 | 10.00 |
| Crospovidone (other alternative is sodium starch | 24.00 | 2.00 |
| glycolate or cross-linked carboxymethylcellulose | | |
| sodium) | | |
| Talc | 24.00 | 2.00 |
| Silicon dioxide | 6.00 | 0.50 |
| Stearic acid | 6.00 | 0.50 |
| Sucralose | 6.00 | 0.50 |
| Total | 1200.00 | 100.00 |

b) Formulation J

| | Amount | |
|---|---|---|
| | (mg/ | |
| Component | tablet) | (%) |
| TM Acetaminophen (250 mg acetaminophen with | 300.00 | 23.08 |
| 50 mg cellulose acetate) | | |
| TM Acetylsalicylic acid (250 g acetylsalicylic acid | 287.50 | 22.12 |
| with 37.5 mg cellulose acetate) | | |
| TM Caffeine (65 mg caffeine with 26 mg cellulose | 91.00 | 7.00 |
| acetate) | | |
| Mannitol | 420.00 | 32.31 |
| Maize starch | 130.00 | 10.00 |
| Crospovidone (other alternative is sodium starch | 26.00 | 2.00 |
| glycolate or cross-linked carboxymethylcellulose | | |
| sodium) | | |
| Talc | 26.00 | 2.00 |
| Silicon dioxide | 6.50 | 0.50 |
| Stearic acid | 6.50 | 0.50 |
| Sucralose | 6.50 | 0.50 |
| Total | 1300.00 | 100.00 |

For both formulations I and J, acetylsalicylic acid drug release from the ODTs is above 70% after 15 minutes when tested using an USP Type II dissolution apparatus (paddle) operating at 100 rpm in 900 ml: of water at 37° C. Acetaminophen and caffeine releases from the ODTs are above 80% after 15 minutes when tested following the same method as described for acetylsalicylic acid (see below dissolution profile of formulation I ODTs).

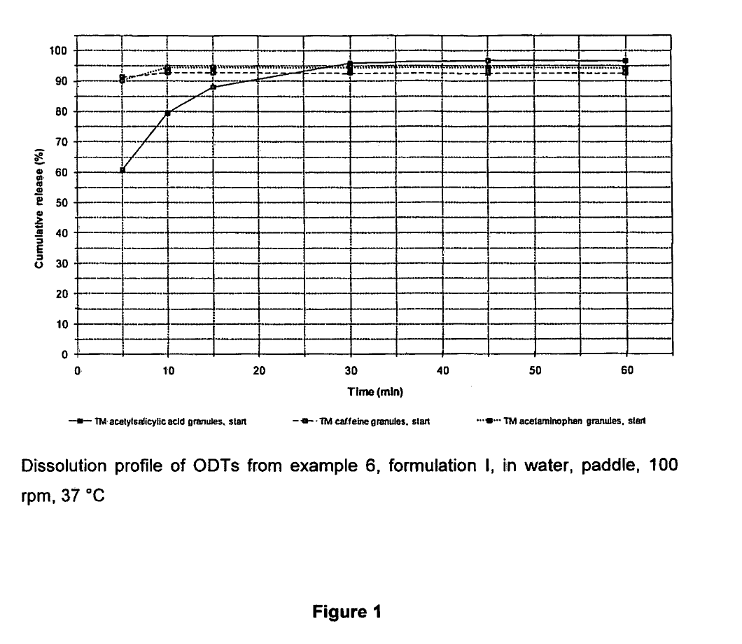

The dissolution profile of formulation I in water, paddle, 100 rpm, 37° C. is shown in FIG. 1.

As it can be seen in table below formulation I ODTs exhibit good purity stability up to 3 months at 25° C./60% rh and at 30° C./65% rh.

Assay, related substances of Formulation I

| | Content | | | Assay | | | Related Substances | | |
|---|---|---|---|---|---|---|---|---|---|
| | Acetamin- | Caf- | Acetylsali- | Acetamin- | Caf- | Acetylsali- | O,N-diacetyl- | Salicyl- | |
| Storage | ophen | feine | cylic acid | ophen | feine | cylic acid | 4-aminophenol | ic Acid | Sum |
| conditions | (%) | (%) | (%) | (%) | (%) | (%) | (%) | (%) | (%) |
| Start values | 16.3 | 4.1 | 20.6 | 97.5 | 97.2 | 98.7 | 0.000 | 0.149 | 0.149 |
| 3 months at | 17.1 | 4.2 | 21.5 | 102.8 | 101.3 | 103.1 | 0.008 | 0.702 | 0.710 |
| 25° C./60% rh | | | | | | | | | |
| 3 months at | 17.0 | 4.3 | 21.4 | 102.1 | 102.2 | 102.9 | 0.014 | 1.085 | 1.099 |
| 30° C./65% rh | | | | | | | | | |

### EXAMPLE 7

### tm Caffeine, tm Acetaminophen, Acetylsalicylic Acid, Mannitol)

Acetaminophen is taste-masked with CA 398-10 (pure cellulose acetate from Eastman) by top-spray processing or Wurster coating (Glatt GPCG 1). Application of the taste-masking agent CA 398-10 is performed by spraying 1.5 kg acetone/water 95:5 solution containing 10% w/w CA 398-10 onto 750.0 g acetaminophen using a spray rate of 25-27 g/min and 25° C. product temperature. After drying (30 minutes at 60° C. inlet air temperature), the taste-masked acetaminophen exhibits a narrow particle size distribution ranging from 50 to 315 μm, with a mean particle size of approximately 1.25 μm.

The taste-masking of caffeine is performed following the same processing conditions as described above for acetaminophen using a solution of acetone/water 95:5 containing 10% w/w CA 398-10. In this case, the application is performed by spraying 3.0 kg acetone/water 95:5 solution containing 10% w/w CA 398-10 onto 750.0 g caffeine with a spray rate of 24-27 g/min and 25° C. product temperature. After drying (30 minutes at 60° C. inlet air temperature), the taste-masked caffeine exhibits a narrow particle size distribution ranging from 50 to 315 μm, with a mean particle size of approximately 125 μm.

The obtained taste-masked micro-products (acetaminophen and caffeine) are combined with acetylsalicylic acid, mannitol, glucose monohydrate, maize starch, crospovidone, sodium starch glycolate or cross-linked carboxymethylcellulose sodium, talc, silicon dioxide, comminuted sucralose, eventually flavors and pigments, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK0 instrumented single station press equipped with 18 mm round, biplane punches, with bevelled edges. The obtained ODTs exhibit an average mass of ˜1200 mg, a crushing strength of ˜40 N and an average height of ˜5 mm.

The disintegration time of the orally disintegrating tablets as described above determined in water according to the method outlined in a) European Pharmacopoeia, Vol. 6, 2.9.1., Apparatus A, is below 60 seconds.

a) Formulation K

| | Amount | |
|---|---|---|
| Component | (mg/tablet) | (%) |
| TM Acetaminophen (200 mg acetaminophen | 240.00 | 20.00 |
| with 40 mg cellulose acetate) | | |
| Acetylsalicylic acid | 250.00 | 20.83 |
| TM Caffeine (50 mg caffeine with 20 mg | 70.00 | 5.83 |
| cellulose acetate) | | |
| Mannitol | 394.00 | 32.83 |
| Glucose monohydrate | 60.00 | 5.00 |
| Maize starch | 120.00 | 10.00 |
| Crospovidone (other alternative is sodium starch | 24.00 | 2.00 |
| glycolate or cross-linked carboxymethylcellulose | | |
| sodium) | | |
| Talc | 24.00 | 2.00 |
| Silicon dioxide | 6.00 | 0.50 |
| Stearic acid | 6.00 | 0.50 |
| Sucralose | 6.00 | 0.50 |
| Total | 1200.00 | 100.00 |

b) Formulation L

| | Amount | |
|---|---|---|
| Component | (mg/tablet) | (%) |
| TM Acetaminophen (250 mg acetaminophen | 300.00 | 25.00 |
| with 50 mg cellulose acetate) | | |
| Acetylsalicylic acid | 250.00 | 20.83 |
| TM Caffeine (65 mg caffeine with 26 mg | 91.00 | 7.58 |
| cellulose acetate) | | |
| Mannitol | 313.00 | 26.08 |
| Glucose monohydrate | 60.00 | 5.00 |
| Maize starch | 120.00 | 10.00 |
| Crospovidone (other alternative is sodium starch | 24.00 | 2.00 |
| glycolate or cross-linked carboxymethylcellulose | | |
| sodium) | | |
| Talc | 24.00 | 2.00 |
| Silicon dioxide | 6.00 | 0.50 |
| Stearic acid | 6.00 | 0.50 |
| Sucralose | 6.00 | 0.50 |
| Total | 1200.00 | 100.00 |

For both formulations K and L, the ODTs exhibit complete acetylsalicylic acid drug release after 5 minutes when tested using an USP Type II dissolution apparatus (paddle) operating at 100 rpm in 900 ml of water at 37° C. Acetaminophen and caffeine releases from the above ODTs are above 80% after 15 minutes when tested following the same method as described for acetylsalicylic acid (see below dissolution profile of formulation K ODTs).

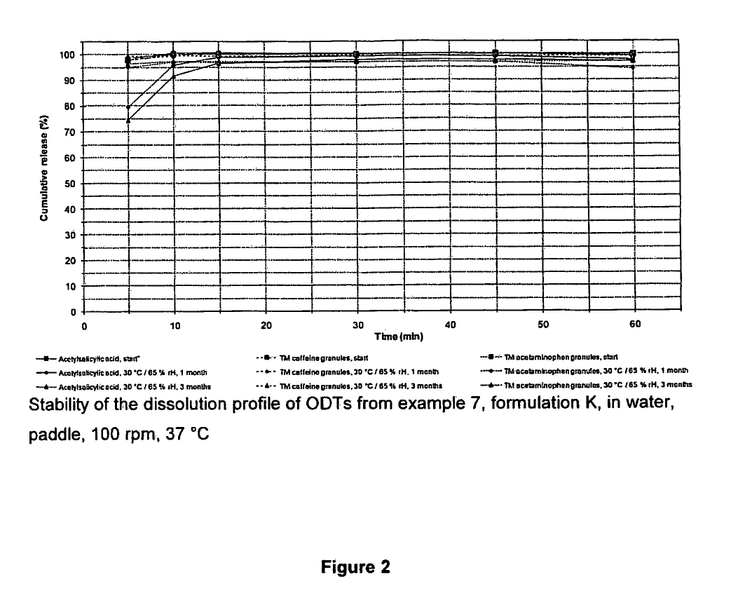

The stability of the dissolution profile of formulation K in water, paddle,. 100 rpm, 37 C is shown in FIG. 2.

Formulation K ODTs exhibit good purity up to 6 months at 25° C./60% rh and at 30° C./65% rh and good dissolution profile stability up to 3 months at 30° C./65% rh.

| | Content | | | Assay | | | Related Substances | | |
|---|---|---|---|---|---|---|---|---|---|
| | Acetamin- | Caf- | Acetylsali- | Acetamin- | Caf- | Acetylsali- | Acetamin- | Caf- | Acetylsali- |
| Storage | ophen | feine | cylic acid | ophen | feine | cylic acid | ophen | feine | cylic acid |
| conditions | (%) | (%) | (%) | (%) | (%) | (%) | (%) | (%) | (%) |
| Start values | 15.8 | 3.9 | 21.5 | 94.9 | 94.8 | 103.3 | 0.000 | 0.148 | 0.148 |
| 6 months at | 15.7 | 3.9 | 20.8 | 94.3 | 93.9 | 100.1 | 0.000 | 0.553 | 0.553 |
| 25° C./60% rh | | | | | | | | | |
| 6 months at | 16.2 | 4.1 | 20.5 | 97.1 | 98.9 | 98.4 | 0.000 | 1.233 | 1.233 |
| 30° C./65% rh | | | | | | | | | |

### EXAMPLE 8

### tm Caffeine, tm Acetaminophen, Acetylsalicylic Acid, Mannitol

Acetaminophen is taste-masked with CA 398-10 (pure cellulose acetate from Eastman) by top-spray processing or Wurster coating (Glatt GPCG 1). Application of the taste-masking agent CA 398-10 is performed by spraying 1.5 kg acetone/water 95:5 solution containing 10% w/w CA 398-10 onto 750.0 g acetaminophen using a spray rate of 21-23 g/min and 25° C. product temperature. After drying (30 minutes at 60° C. inlet air temperature), the taste-masked acetaminophen exhibits a narrow particle size distribution ranging from 50 to 31.5 μm, with a mean particle size of approximately 125 μm.

The taste-masking of caffeine is performed following the same processing conditions as described: above for acetaminophen using a solution of acetone/water 95:5 containing 10% w/w CA 398-10. In this case, the application is performed by spraying 3.0 kg acetone/water 95:5 solution containing 10% w/w CA 398-10 onto 750.0 g caffeine with a spray rate of 22-31 g/min and 25° C. product temperature. After drying (30 minutes at 60° C. inlet air temperature), the taste-masked caffeine exhibits a narrow particle size distribution ranging from 50 to 315 μm, with a mean particle size of approximately 125 μm.

The obtained taste-masked micro-products (acetaminophen and caffeine) are combined with acetylsalicylic acid, mannitol, microcrystalline cellulose, crospovidone, sodium starch glycolate or cross-linked carboxymethylcellulose sodium, talc, comminuted sucralose, eventually flavors and pigments, and pre-blended during 15 minutes in a Turbula T2C diffusion blender. After pre-blending, stearic acid is added to the pre-blend and blending is continued with the same equipment for additional 5 minutes.

Compression of laboratory scale batches is performed with a Korsch EK0 instrumented single station press equipped with 18 mm round, biplane punches, with bevelled edges. The obtained ODTs exhibit an average mass of ˜1200 mg, a crushing strength of ˜40 N and an average height of ˜5 mm.

The disintegration time of the orally disintegrating tablets as described above determined in water according to the method outlined in a) European Pharmacopoeia, Vol. 6, 2.9.1., Apparatus A, is below 60 seconds.

a) Formulation M

| | Amount | |
|---|---|---|
| Component | (mg/tablet) | (%) |
| TM Acetaminophen (200 mg acetaminophen | 240.00 | 20.00 |
| with 40 mg cellulose acetate) | | |
| Acetylsalicylic acid | 250.00 | 20.83 |
| TM Caffeine (50 mg caffeine with 20 mg | 70.00 | 5.83 |
| cellulose acetate) | | |
| Mannitol | 496.00 | 41.33 |
| Microcristalline cellulose | 12.00 | 1.00 |
| Crospovidone (other alternative is sodium starch | 84.00 | 7.00 |
| glycolate or cross-linked carboxymethylcellulose | | |
| sodium) | | |
| Talc | 36.00 | 3.00 |
| Stearic acid | 6.00 | 0.50 |
| Sucralose | 6.00 | 0.50 |
| Total | 1200.00 | 100.00 |

b) Formulation N

| | Amount | |
|---|---|---|
| Component | (mg/tablet) | (%) |
| TM Acetaminophen (250 mg acetaminophen | 300.00 | 25.00 |
| with 50 mg cellulose acetate) | | |
| Acetylsalicylic acid | 250.00 | 20.83 |
| TM Caffeine (65 mg caffeine with 26 mg | 91.00 | 7.58 |
| cellulose acetate) | | |
| Mannitol | 415.00 | 34.58 |
| Microcristalline cellulose | 12.00 | 1.00 |
| Crospovidone (other alternative is sodium starch | 84.00 | 7.00 |
| glycolate or cross-linked carboxymethylcellulose | | |
| sodium) | | |
| Talc | 36.00 | 3.00 |
| Stearic acid | 6.00 | 0.50 |
| Sucralose | 6.00 | 0.50 |
| Total | 1200.00 | 100.00 |

For both formulations M and N, the ODTs exhibit complete acetylsalicylic acid drug release after 5 minutes when tested using an USP Type II dissolution apparatus (paddle) operating at 100 rpm in 900 ml of water at 37° C. Acetaminophen and caffeine releases from the above ODTs are above 80% after 15 minutes when tested following the same method as described for acetylsalicylic acid (see below dissolution profile of formulation M ODTs).

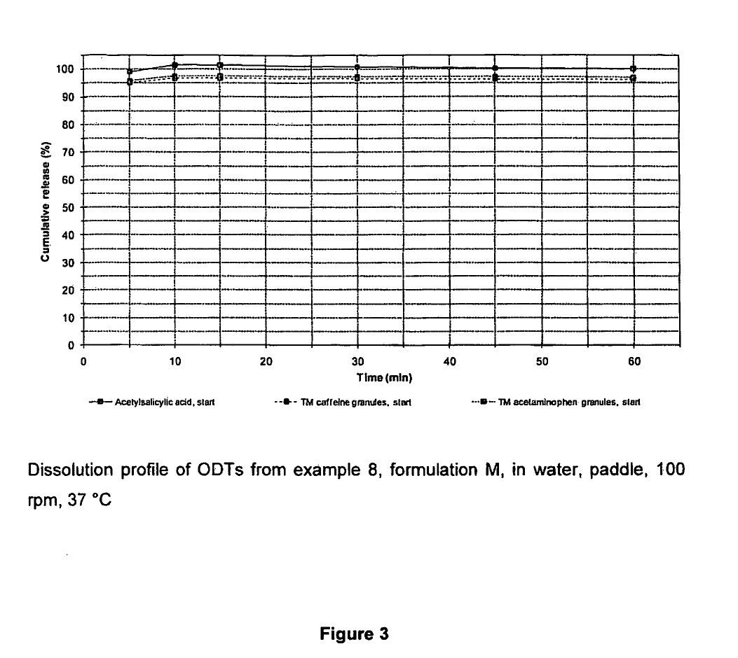

The dissolution profile of formulation M in water, paddle, 100 rpm, 37° C. is shown in FIG. 3.

As it can be seen below formulation M ODTs exhibit good purity stability up to 3 months at 25° C./60% rh and at 30° C./65% rh.

| | Content | | | Assay | | | Related Substances | | |
|---|---|---|---|---|---|---|---|---|---|
| | Acetamin- | Caf- | Acetylsali- | Acetamin- | Caf- | Acetylsali- | O,N-diacetyl- | Salicyl- | |
| Storage | ophen | feine | cylic acid | ophen | feine | cylic acid | 4-aminophenol | ic Acid | Sum |
| conditions | (%) | (%) | (%) | (%) | (%) | (%) | (%) | (%) | (%) |
| Start values | 16.7 | 4.2 | 21.1 | 100.1 | 100.0 | 101.1 | 0.000 | 0.122 | 0.122 |
| 3 months at | 16.7 | 4.1 | 21.0 | 99.9 | 98.3 | 100.9 | 0.000 | 0.300 | 0.300 |
| 25° C./60% rh | | | | | | | | | |
| 3 months at | 16.8 | 4.2 | 20.9 | 100.8 | 100.2 | 100.2 | 0.004 | 0.406 | 0.410 |
| 30° C./65% rh | | | | | | | | | |

### EXAMPLE 9

### tm Caffeine, tm Acetaminophen, Acetylsalicylic Acid, Mannitol

Acetaminophen is taste-masked with CA 398-10 (pure cellulose acetate from Eastman) by top-spray processing or Wurster coating (Glatt GPCG 1). Application of the taste-masking agent CA 398-10 is performed by spraying 1.5 kg acetone/water 95:5 solution containing 10% w/w CA 398-10 onto 750.0 g acetaminophen using a spray rate of 21-22 g/min and 25° C. product temperature. After drying (30 minutes at 60° C. inlet air temperature), the taste-masked acetaminophen exhibits a narrow particle size distribution ranging from 50 to 315 μm, with a mean particle size of approximately 125 μm.

The taste-masking of caffeine is performed following the same processing conditions as described above for acetaminophen using a solution of acetone/water 95:5 containing. 10% w/w CA 398-10. In this case, the application is performed by spraying 3.0 kg acetone/water 95:5 solution containing 10% w/w CA 398-10 onto 750.0 g caffeine with a spray rate of 22-31 g/min and 25° C. product temperature. After drying (30 minutes at 60° C. inlet air temperature), the taste-masked caffeine exhibits a narrow particle size distribution ranging from 50 to 315 μm, with a mean particle size of approximately 125 μm.

The obtained taste-masked. micro-products (acetaminophen and caffeine) are combined with acetylsalicylic acid, mannitol, glucose monohydrate, microcrystalline cellulose, maize starch, crospovidone, sodium starch glycolate or cross-linked carboxymethylcellulose sodium, eventually communited sucralose, flavors and pigments, and pre-blended during 15 minutes in a Turbula T2C diffusion blender.

Compression of laboratory scale batches is performed with a Korsch EK0 instrumented single station press equipped with 18 mm round, biplane punches, with bevelled edges. Before each compression cycle talc is applied with a pencil on the upper and lower punches to prevent sticking phenomenon between tablets faces and punches surfaces. The obtained ODTs exhibit an average mass of ˜1200 mg, a crushing strength of ˜40 N and an average height of ˜5 mm.

Production scale batches can be performed with a Fette rotary press equipped with PKB system to perform the punch lubrication with suitable lubricant/anti-tacking agent/glidants.

The disintegration time of the orally disintegrating tablets as described above determined in water according to the method outlined in a) European Pharmacopoeia, Vol. 6, 2.9.1., Apparatus A, is below 60 seconds.

a) Formulation O

| | Amount | |
|---|---|---|
| Component | (mg/tablet) | (%) |
| TM Acetaminophen (200 mg acetaminophen with | 240.00 | 20.00 |
| 40 mg cellulose acetate) | | |
| Acetylsalicylic acid | 250.00 | 20.83 |
| TM Caffeine (50 mg caffeine with 20 mg | 70.00 | 5.83 |
| cellulose acetate) | | |
| Mannitol | 412.00 | 34.33 |
| Glucose monohydrate | 60.00 | 5.00 |
| Microcristalline cellulose | 24.00 | 2.00 |
| Maize starch | 120.00 | 10.00 |
| Crospovidone | 24.00 | 2.00 |
| Total | 1200.00 | 100.00 |

b) Formulation P

| | Amount | |
|---|---|---|
| Component | (mg/tablet) | (%) |
| TM Acetaminophen (250 mg acetaminophen with | 300.00 | 25.00 |
| 50 mg cellulose acetate) | | |
| Acetylsalicylic acid | 250.00 | 20.83 |
| TM Caffeine (65 mg caffeine with 26 mg | 91.00 | 7.58 |
| cellulose acetate) | | |
| Mannitol | 331.00 | 27.58 |
| Glucose monohydrate | 60.00 | 5.00 |
| Microcristalline cellulose | 24.00 | 2.00 |
| Maize starch | 120.00 | 10.00 |
| Crospovidone | 24.00 | 2.00 |
| Total | 1200.00 | 100.00 |

For both formulations O and P, the ODTs exhibit complete acetylsalicylic acid drug release after 5 minutes when tested using an USP Type Il dissolution apparatus (paddle) operating at 100 rpm in 900 ml of water at 37° C. Acetaminophen and caffeine releases from the above ODTs are above 80% after 15 minutes when tested following the same method as described for acetylsalicylic acid (see below dissolution profile of formulation. O ODTs).

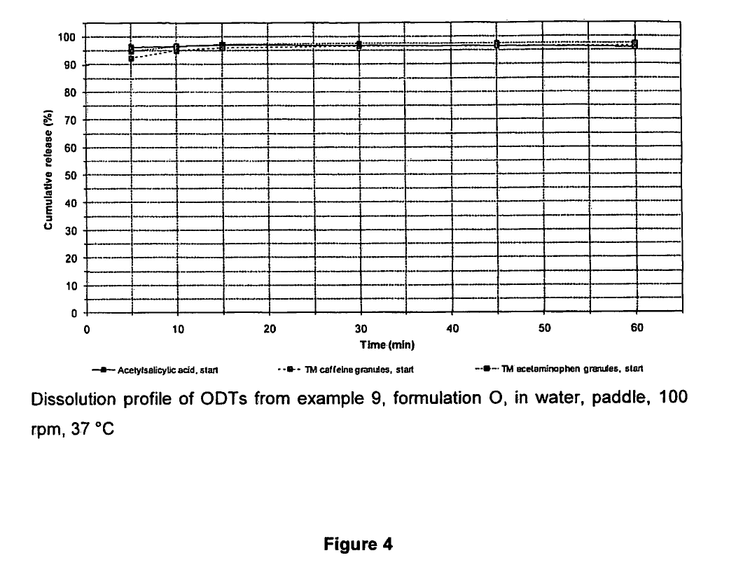

The dissolution profile of formulation O in water, paddle, 100 rpm, 37° C. is shown in FIG. 4.

As it can be seen below formulation O ODTs exhibit good purity stability up to 1 month at 25° C./60% rh and 30° C./65% rh.

| | Content | | | Assay | | | Related Substances/API | | |
|---|---|---|---|---|---|---|---|---|---|
| | Acetamin- | Caf- | Acetylsali- | Acetamin- | Caf- | Acetylsali- | O,N-diacetyl- | Salicyl- | |
| Storage | ophen | feine | cylic acid | ophen | feine | cylic acid | 4-aminophenol | ic Acid | Sum |
| conditions | (%) | (%) | (%) | (%) | (%) | (%) | (%) | (%) | (%) |
| Start values | 16.4 | 4.0 | 20.4 | 98.2 | 96.0 | 98.1 | 0.000 | 0.128 | 0.128 |
| 1 month at | 16.6 | 4.1 | 20.9 | 99.3 | 98.0 | 100.3 | 0.010 | 0.212 | 0.223 |
| 25° C./60% rh | | | | | | | | | |
| 1 month at | 16.7 | 4.1 | 21.1 | 100.3 | 98.4 | 101.2 | 0.016 | 0.274 | 0.291 |
| 30° C./65% rh | | | | | | | | | |

### REFERENCES CITED

[1] USP 31/NF 26, 2008

[2] Martindale, The Extra Pharmacopoeia, 31^{st} edition, 1996

[3] www.thomapyrin.de

[4] www.excedrin.com

[5] European Pharmacopoeia, 6^{th} edition, 2007

[6] Diener, H. C. et al, Cephalalgia, Volume 25, Issue 10, Pages 776-787

[7] Guidance for Industry, Orally Disintegrating Tablets, December 2008

[8] Bandar, S. et al, Asian Journal of Pharmaceutics, January 2008

[9] Pfister, W. R. et al., Pharmaceutical technology, Oct. 1, 2005

1. An orally disintegrating tablet comprising acetylsalicylic acid, acetaminophen and caffeine and one or more pharmaceutically acceptable excipients.

2. The orally disintegrating tablet according to claim 1, comprising 50 to 1000 mg acetylsalicylic acid, 50 to 1000 mg acetaminophen and 20-200 mg caffeine.

3. The orally disintegrating tablet according to claim 1, comprising 250 mg acetylsalicylic acid, 200 mg acetaminophen and 50 mg caffeine.

4. The orally disintegrating tablet according to claim 1, comprising 250 mg acetylsalicylic acid, 250 mg acetaminophen and 65 mg caffeine.

5. The orally disintegrating tablet according to claim 1, comprising a taste-masking agent.

6. The orally disintegrating tablet according to claim 1, wherein the acetaminophen is contained in the form of micro-particles comprising a taste-masking coating, and/or wherein the caffeine is contained in the form of micro-particles comprising a taste-masking coating.

7. The orally disintegrating tablet according to claim 6, wherein the acetylsalicylic acid is contained in the form of micro-particles comprising a taste-masking coating.

8. The orally disintegrating tablet according to claim 6, wherein the taste-masking coating comprises a polymer selected from the group consisting of ethyl cellulose and cellulose acetate.

9. The orally disintegrating tablet according to claim 1, comprising spherical particles comprising mannitol and a disintegrant.

10. The orally disintegrating tablet according to claim 9, wherein the disintegrant is selected from crospovidone, sodium starch glycolate, cross-linked sodium carboxymethyl cellulose, and any mixture thereof.

11. The orally disintegrating tablet according to any of claims 1 comprising acetylsalicylic acid, acetaminophen micro-particles having a taste-masking coating comprising cellulose acetate, caffeine microparticles having a taste-masking coating comprising cellulose acetate, mannitol and a disintegrant.

12. A method for preparing an orally disintegrating tablet according to claim 1, comprising a) blending the acetylsalicylic acid, acetaminophen and caffeine with the one or more pharmaceutically acceptable ingredients, and b) compressing the resulting blend into tablets.
